(19) United States
(12) Patent Application Publication
(10) Pub. No.: US 2020/0375508 A1
(43) Pub. Date: Dec. 3, 2020

(54) DEVICE AND METHOD FOR DETERMINING THE FREQUENCY OF AT LEAST ONE DOMINANT TONE OF A TINNITUS

(71) Applicant: Forschungszentrum Juelich GmbH, Juelich (DE)

(72) Inventors: Peter Alexander Tass, Tegernsee (DE); Christian Hauptmann, Starnberg (DE)

(21) Appl. No.: 16/845,169

(22) Filed: Apr. 10, 2020

Related U.S. Application Data
(63) Continuation of application No. 15/533,767, filed on Jun. 7, 2017, now abandoned.

(30) Foreign Application Priority Data
Dec. 15, 2014 (DE) 10 2014 118674.5

(51) Int. Cl. A61B 5/12

(52) U.S. Cl. CPC A61B 5/128; A61F 11/00; A61B 5/121

(57) ABSTRACT

A device for determining the frequency of a dominant tone of a tinnitus perceived by a patient. A tone generator plays tones, an input unit receives patient evaluations, and a controller carries out a similarity measurement and a comparison of pairs of tones. The controller controls the tone generator for the similarity measurement to generate successive tones, and, after playing a respective tone, receive patient's evaluation regarding the similarity of the tone to the dominant tone of the tinnitus. The controller also controls the tone generator for the comparison of pairs of tones to play successive pairs of tones, and, after playing a respective pair, receives the patient's evaluation as to which tone of the pair is more similar to the at least one dominant tone of the tinnitus. Using the results, the frequency most similar to the frequency of the dominant tone of the tinnitus can be determined.

### CROSS-REFERENCE TO RELATED APPLICATIONS

This current application is a continuation application of U.S. application Ser. No. 15/533,767, filed Jun. 7, 2017, which is a National Stage entry of International Application No. PCT/EP2015/079720, filed Dec. 15, 2015, which claims priority to German Patent Application No. 10 2014 118674.5 filed Dec. 15, 2014, the disclosures of these priority applications are incorporated in their entirety herein by reference.

### TECHNICAL FIELD

The invention relates to an apparatus and to a method of determining the frequency of at least one dominant sound of a ringing in the ear perceived by a patient.

### BACKGROUND

Subjective tinnitus is the perception of a sound or noise without physical source of sound or source of noise. The subjective tinnitus arises in the brain of the patient and can only be perceived by him. It is a wide spread disease which has a torturous character for many patients. One differentiates between a tonal tinnitus, this means the perception of one or more sounds in comparison to an atonal tinnitus, this means the perception of a noise. The noise perceived for an atonal tinnitus can however, also include one or more dominant sounds.

In particular with regard to the application of the acoustic “coordinated reset” for neuromodulation (CR-neuromodulation) for the therapy of the tinnitus, it is extremely important to precisely determine the frequencies, this means the position of the pitch of the sound of the dominant sounds as precisely as possible.

So far methods were used for the determination of the tinnitus frequency in which the patient was typically played a comparative sound and the patient could set the frequency and the amplitude of the comparative sound himself in such a way that the comparative sound corresponded to the tinnitus sound. These methods are tedious and for a relevant number of patients lead to non-reproducible and in this way to non-reliable results (see E. M. Burns: A Comparison of Variability among Measurements of Subjective Tinnitus and Objective Stimuli, Audiology 23, 426-440 (1984)).

### SUMMARY

For this reason it is an object of the invention to provide an apparatus as well as a method that enable the determination of frequency of at least one dominant sound of a ringing in the ear perceived by a patient with a large accuracy and a high probability of reproducibility.

The object underlying the invention is satisfied by the features of the independent claims. Advantageous further embodiments and designs of the invention are stated in the subordinate claims.

### BRIEF DESCRIPTION OF THE DRAWINGS

The invention will be described in detail in the following in an exemplary manner with reference to the drawing. In this is shown:

FIG. 1 illustrates a schematic illustration of an apparatus for determining the frequency of at least one dominant sound of a ringing in the ear perceived by a patient;

FIG. 2 illustrates a table for inputting data obtained for determining the frequency of the at least one dominant sounds; and

FIG. 3 illustrates a table filled with exemplary data for determining the frequency of the at least one dominant sound.

### DETAILED DESCRIPTION

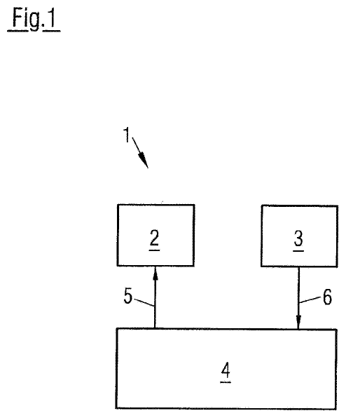

In FIG. 1 an apparatus 1 for determining the frequency of at least one dominant sound of a ringing in the ear perceived by a patient is schematically illustrated. The apparatus 1 is composed by a sound generator 2, an input unit 3 and a control and analysis unit 4 coupled to the sound generator 2 as well as to the input unit 3.

The sound generator 2 generates sounds that are played to a patient suffering from tinnitus. The sounds are comparative sounds that the patient compares to the dominant sound to be investigated of the ringing in the ear perceived by him and correspondingly evaluates these.

The evaluation of the provided sounds carried out by the patient is input into the input unit 3.

The control and analysis unit 4 during the operation of the apparatus 1 carries out a control of the sound generator 2. For this purpose the control and analysis unit 4 transmits control signals 5 to the sound generator 2. Furthermore, the control and analysis unit 4 receives signals 6 from the input unit 3 by means of which the control and analysis unit 4 is transmitted evaluations from the patient input into the input unit 3 with respect to the sounds made available to the patient.

The control and analysis unit 4 with the aid of the sound generator 2 and of the input unit 3 carries out different investigations, in particular a similarity measurement and a pairwise sound comparison.

Having regard to the similarity measurement the control and analysis unit 4 controls the sound generator 2 in such a way that this plays different sounds to the patient one after the other. After each played sound, the patient evaluates the respective sound with respect to the similarity of the sound to the dominant sound of the ringing in the ear perceived by the patient to be investigated. The evaluation of the patient is input into the input unit 3 and is forwarded to the control and analysis unit 4.

In accordance with a design the sounds that are played to the patient during the similarity measurement one after the other are spaced apart from one another in frequency, this means that a frequency ramp is not raised and or reduced for the similarity measurement.

With respect to the pairwise sound comparison, the control and analysis unit 4 controls the sound generator 2 in such a way that this plays pairs of sounds one after the other to the patient. The sounds of a respective pair are in this respect made available delayed in time and in particular in a non-overlapping manner. After each pair, the patient evaluates which sound of the pair is more similar to the dominant sound of the ringing in the ear to be investigated. The evaluation of the patient is input into the input unit 3 and is forwarded to the control and analysis unit 4.

The control and analysis unit 4 combines the results of the similarity measurement and of the pairwise sound comparison and therefrom determines at least one sound whose frequency is most similar to the frequency of the dominant sound of the ringing in the ear to be investigated.

The ringing in the ear perceived by the patient that is investigated with the apparatus 1 can both be a tonal as well as an atonal tinnitus. In the case of tonal tinnitus the patient perceives at least one sound whose frequency can be determined with the aid of the apparatus 1. In the case of a atonal tinnitus the patient perceives a noise which can include at least one dominant sound whose frequency is determined by the apparatus 1. In the event that the patient perceives a plurality of dominant sounds whose frequencies shall be determined, then the similarity measurement and the pairwise sound comparison have to be carried out for each of the dominant sounds.

The sounds played to the patient can be described mathematically by means of a sinus oscillation. Each sound is characterized by a pitch, this means the frequency of the sinus oscillation, and a volume, this means the amplitude of the sinusoidal oscillation. Pure sounds can be generated by the sound generator 2 whose frequency bandwidth is ideally very small. The frequency bandwidth in practice is limited by the devices generating the sounds. Furthermore, also sounds can be used for the investigations described herein that have a predefined frequency bandwidth.

The sound generator 2 generating the sounds can have earphones or also headphones or loudspeakers that are configured in a different way, wherein an earphone is a loudspeaker placed into the ear canal. Furthermore, the sound generator 2 can have an electric circuit for the generation of the sounds. The sounds can be made available for the patient at an ear or at both ears.

Having regard to a one-sided tinnitus the sounds are made available by the sound generator 2 at the same ear and/or at the opposite ear. With respect to two-sided tinnitus the sounds are applied at the same ear and/or at the opposite ear of the dominant sound of a tinnitus. Having regard to identical at both sides and/or centrally perceived tinnitus at both sides the sounds are made available by the sound generator 2 at one side and/or at both sides.

The control and analysis unit 4 can include a processing unit for carrying out its tasks, e.g. a processor or a microcontroller. Furthermore, the control and analysis unit 4 can be a computer. The herein described method for determining the frequency of the at least one dominant sound of the ringing in the ear perceived by the patient can be stored as software code in a memory associated with the control and analysis unit 4.

The input unit 3 can e.g. be the keyboard of a computer or it can also comprise a menu illustrated on a screen via which the inputs can be affected. Typically, the patient provides the evaluations of the sounds to a doctor or audiologist which the doctor or the audiologist respectively then inputs into the input unit 3. It would also be plausible that the patient inputs the evaluations himself into the input unit 3.

In accordance with an embodiment the control and analysis unit is configured to determine at least one sound as a result of the similarity measurement whose frequency is most similar to the frequency of the dominant sound of the ringing in the ear to be investigated in accordance with the similarity measurement. Furthermore, the control and analysis unit 4 determines at least one sound as a result of the pairwise sound comparison whose frequency is most similar to the frequency of the dominant sound to be investigated of the ringing in the ear in accordance with the pairwise sound comparison.

Having regard to the similarity measurements a similarity value can be determined for each provided sound which states the similarity of the respective sound to the dominant sound of the ringing in the ear at a predefined scale. Subsequently the sound with the highest similarity value can be selected as that sound that is most similar to the dominant sound of the ringing in the ear according to the similarity measurement. It is also plausible to not only select one sound, but a plurality of sounds that have the highest similarity values at the end of the similarity measurement.

An embodiment provides that, for a pairwise sound comparison, that sound is selected from each pair of sounds played to the patient that is most similar to the dominant sound of the ringing in the ear in accordance with the estimate of the patient. Such comparisons are carried out for a predefined number of pairs. The pairs are configured in accordance with an embodiment in such a way that the frequency spacing determined over all pairs, in particular the relative frequency spacing between the two sounds of a pair, is as large as possible. As a consequence thereof in particular pairs of non-adjacent sounds are formed. Subsequently new pairs of the previously selected sounds are formed and the sounds of these pairs are compared again to the dominant sound of the ringing in the ear by the patient and the more similar sounds are selected. This method can correspondingly be carried on further up until one sound remains that is considered as that sound that is most similar to the dominant sound of the ringing in the ear in accordance with the pairwise sound comparison. Also in this case it can be provided to select more than one sounds at the end of the investigation and not only one sound.

The similarity measurement and the pairwise sound comparison can be carried out independent from one another. The results of the two investigations can subsequently be combined with one another in a suitable manner in order to determine the frequency of the dominant sound of the ringing in the ear perceived by the patient.

Alternatively, the similarity measurement and the pairwise sound comparison can be carried out by the control and analysis unit 4 in a predefined sequence. The result of the investigation carried out first can then be used as a starting value for the subsequent investigation. In this way, for example, the similarity measurement can be carried out as a first investigation and the results of the similarity measurement can be used for the purpose of determining the starting sounds of the pairwise sound comparison. In particular, in this case, sounds can exclusively be played to the patient during the pairwise sound comparison that lie within a predefined frequency range lying around the frequency of the sound determined as a result of the similarity measurement having the largest similarity to the dominant sound of the ringing in the ear. From this frequency range sounds can be selected for the pairwise comparison in accordance with an embodiment and pairs can be formed from the selected sounds in such a way that the frequency spacing determined for all pairs, in particular the relative frequency spacing between the two pairs of a pair, is as large as possible.

In the reverse case, in which the similarity measurement can follow the pairwise sound comparison, the result of the pairwise sound comparison can be drawn upon as the starting value for the similarity measurement.

The similarity measurement and the pairwise sound comparison can provide the starting value or starting region of the respective other investigation. However, they can also provide more reliable information also by way of a redundant test.

Both with respect to the similarity measurement and also with respect to the pairwise sound comparison a frequency spacing between adjacent sounds can respectively be predefined. The investigation carried out first then typically has a larger frequency spacing, than the investigation carried out thereafter. In the event that the similarity measurement is carried out first and then the pairwise sound comparison is carried out, sounds lying adjacent to one another with respect to their frequency of the pairwise sound comparison lie closer together than adjacent sounds of the similarity measurement.

In accordance with an embodiment of the aforementioned design, a coarse estimate of the tinnitus can be carried out with the aid of predefined test sounds before the similarity measurement and the pairwise sound comparison. For this purpose the predefined test sounds can be played one after the other to the patient and the patient states whether the frequency of the respective test sound is larger or smaller than the frequency of the dominant sound of the ringing in the ear. The frequency spacing between adjacent test sounds is larger for this test than the frequency spacing between adjacent sounds of the similarity measurement and of the pairwise sound comparison. Furthermore, following the similarity measurement and the pairwise sound comparison, a fine adjustment of the sound can be carried out which has become apparent from the similarity measurement and the pairwise sound comparison as that sound that is most similar to the at least one dominant sound of the ringing in the ear. The fine adjustment can e.g. be carried out in the boundaries of ±10% around the determined sound. The frequency resolution during the fine adjustment is smaller than for the similarity measurement and the pairwise sound comparison.

During the similarity measurement and/or the pairwise sound comparison the similarity of the sounds can be evaluated by the patient by means of a psychophysical degree of similarity, in particular with respect to the pitch and/or the volume and/or the characteristic of the sound.

In accordance with an embodiment, the similarity measurement consists of a sequence of similarity measurements, wherein the sequence of the individual similarity test depends on the respective previous results. In particular a respective sound that is played to the patient for a similarity test can depend on the evaluation carried out by the patient of one or more previously played sounds.

In analogy thereto a sound comparison can consist of a sequence of pairwise sound comparisons whose sequence depends on the respective previous results. In particular a respective pair of sounds that is played to the patient for the pairwise comparison depends on the evaluation carried out by the patient of one or more previously played pairs of sounds.

Moreover, such sequences of individual similarity measurements and sound comparisons do not have to be carried out separately, but can also be carried out mixed and, with respect to their sequence, in dependence on the respective previous results.

The amplitude, this means the volume, of the sound played to the patient can be determined in that a test sound is played for the patient and the patient sets the amplitude of this sound in such a way that the played sound is just as loud as the dominant sound of the ringing in the ear perceived by the patient. Subsequently the amplitude of the sounds played for the similarity measurement and/or the pairwise sound comparison can be matched to the amplitude set by the patient.

Furthermore, the amplitude of the sound played for the patient can be determined in that the amplitudes of two different test sounds are set by the patient in such a way that they respectively ring as loud as the dominant sound of the ringing in the ear. The two test sounds can then be used as a supporting source of an interpolation or extrapolation which permits the determination of the amplitude of a respective sound played for the similarity measurement and/or the pairwise sound comparison.

Moreover, the amplitude of a respective sound played for the similarity measurement and/or of the pairwise sound comparison can be determined by comparison to a (in particular selected for all such tests) reference sound.

As a result, the sounds played for the patient for the similarity measurement and/or the pairwise sound comparison can respectively all have nearly the same volume as the dominant sound of the ringing in the ear perceived by the patient. The invention furthermore also permits that one can deviate from this and the volume of the sounds played for the patient for the similarity measurement and/or the pairwise sound comparison is larger or smaller than the volume of the dominant sound of the ringing in the ear perceived by the patient.

Prior to carrying out the similarity measurements and the pairwise sound comparison it is sensible to coarsely limit the frequency range from which the test sounds are selected. For this purpose sounds can be played to the patient one after the other and the patient respectively states whether the frequency of the sound is larger or smaller than the frequency of the dominant sound of the ringing in the ear.

As a result a limited, but yet still comparatively broad frequency range is determined in which the frequency of the dominant sound of the ringing in the ear is present. With respect to the subsequent similarity measurement and the pairwise sound comparison the sounds played to the patient or pairs of sounds are exclusively selected from the determined frequency range.

A method for determining the frequency of at least one dominant sound of a ringing in the ear perceived by a patient comprises that a similarity measurement is carried out during which the patient is played sounds one after the other and the patient evaluates the similarity of the sound to the at least one dominant sound of the ringing in the ear after the playing of a respective sound. Furthermore, a pairwise sound comparison is carried out during which the patient is played pairs of sounds one after the other and after the playing of a respective pair the patient evaluates which sound of the pair is more similar to a dominant sound of the ringing in the ear. The results of the similarity measurement and of the pairwise sound comparison are combined and therefrom at least one sound is determined which frequency is most similar to the frequency of the at least one dominant sound of the ringing in the ear. The afore-described method can be carried out by a doctor or audiologist. The method can comprise all previously described embodiments. For the minimal variant of the method, a sound generator for generating the test sounds is sufficient as a technical aid.

Software is executed in a data processing system, in particular in the control and analysis unit 4. The software effects that control signals for controlling a sound generator for playing a sound are generated, evaluations of the played sounds by a patient are received by an input unit, the sound generator is controlled for a similarity measurement in such a way that this plays sounds one after the other and after the playing of a respective sound an evaluation of the patient with respect to the similarity of the sound to the at least one dominant sound of a ringing in the ear perceived by the patient is received by the input unit. Furthermore, the software effects that the sound generator is controlled for a pairwise sound comparison in such a way that this plays pairs of sounds one after the other and after the playing of a respective pair an evaluation of the patient is received by the input unit which sound of the pair is more similar to the at least one dominant sound of the ringing in the ear and the results of the similarity measurement and of the pairwise sound comparison are combined and therefrom at least one sound is determined whose frequency is most similar to the frequency of the at least one dominant sound of the ringing in the ear. The software can comprise all previously described embodiments described in connection with the apparatus 1.

As a further embodiment a method will be described in the following with which the frequency of the at least one dominant sound of a ringing in the ear perceived by the patient can be determined. The subsequently described method can be carried out with the aid of the apparatus 1 illustrated in FIG. 1. The instructions to the patient carrying out the method can, for example, appear on a screen, it can however also be provided that a doctor or an audiologist gives the instructions to the patient.

Prior to carrying out the actual measurement the meaning of amplitude, this means the volume, and the pitch, this means the frequency, shall be explained to the patient in order to put the patient in the position of being able to differentiate sounds.

To explain the amplitude, a sound of 1,000 Hz is played to the patient. Subsequently the amplitude is increased. For explaining the pitch initially a sound of 1,000 Hz is played and subsequently a sound of 1,800 Hz is played. It is explained to the patient that the second sound (1,800 Hz) has a higher pitch than the first sound (1,000 Hz). Thereafter both sounds are played again.

To check the understanding, a first sound of 2,000 Hz and a second sound of 2,400 Hz are played. The patient is asked which sound has a higher pitch. The test is repeated twice with 3,000 Hz and 1,800 Hz.

Furthermore, a first quiet sound of 2,700 Hz and a second louder sound of 2,700 Hz are played, wherein the volume of both sounds lies above the audible threshold. The patient is asked which sound is louder and whether the patient perceives a difference of the sound frequency or not.

Furthermore, three sounds of 14,000 Hz, 11,200 Hz and 8,900 Hz are played in descending sequence. The patient is asked whether the pitch of the sounds become higher or lower or are similar. If the patient cannot differentiate between the highest pitches these sounds are omitted from the subsequent work steps.

When the check of the understanding is successfully carried out with the patient, the frequency determination of the ringing in the ear can then be started. If not the previous steps of the explanation phase can be carried out again.

The following work steps are separately carried out for the left ear and for the right ear. One starts with the side at which the patient perceives the ringing in the ear as loudest. Furthermore, each comparative sound is played, for example, for at least 2 seconds.

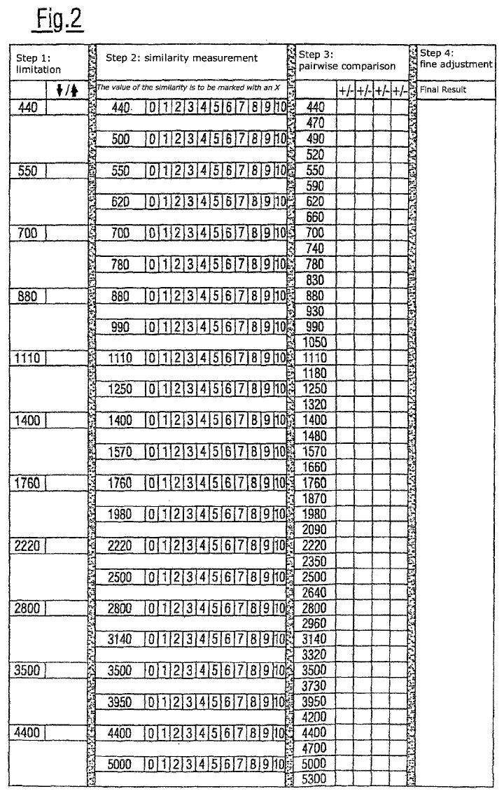

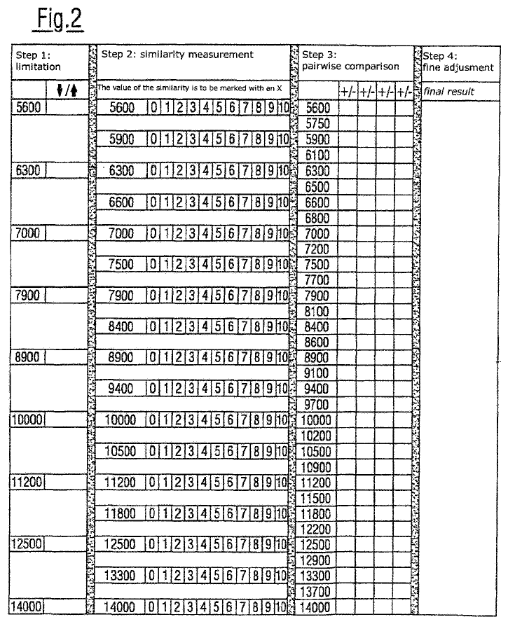

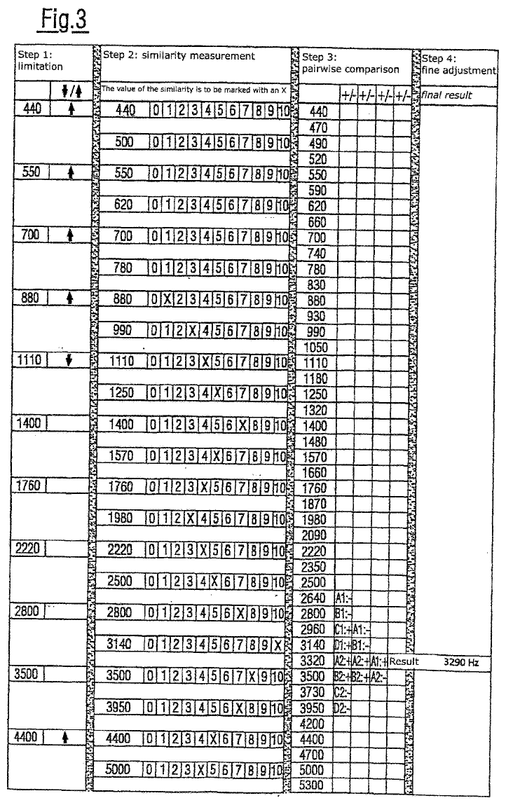

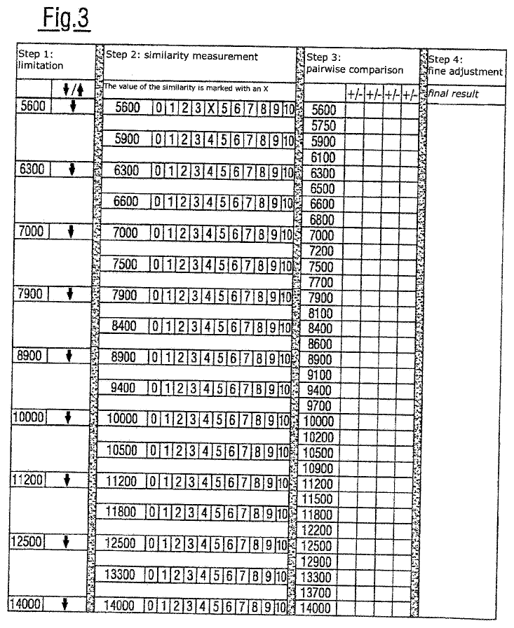

The results of the work steps can be input into a table as is illustrated by way of example in FIG. 2. The table can be filled out by the doctor or the audiologist, it can however, also be stored in a memory to which the control and analysis unit 4 has access, in such a way that the corresponding data can be input into the table by the control and analysis unit 4. For a simpler understanding of the investigations, FIG. 3 shows a table filled with the exemplary data described in the following.

In step 1 a coarse limitation of the frequency of the dominant sound of the ringing in the ear to be investigated takes place. It is recommended to not carry out the coarse limitation on the first investigation in order to determine the complete spectrum of the ringing in the ear of the respective patient. The knowledge of the complete spectrum is very helpful as soon as a first dominant sound of the ring of ear is reduced by a therapy, e.g. a CR neuromodulation and a second dominant sound of the ringing in the ear has to be determined.

The aim of this step is to obtain a first coarse impression of the frequency of the dominant sound of the ringing in the ear of the respective patient. The patient should hear a certain number of test sounds, wherein the patient should be asked whether these lie above or below the frequency of the dominant sound of the ringing in the ear of the patient. For this step 16 sounds are used that cover the frequency ranges between 440 Hz and 14,000 Hz. The step size is a third of an octave (approximately 25% of the relative difference to the next lying test sounds).

Initially the method and its sequence are explained to the patient.

For limiting the spectrum downwardly one starts with the lowest frequency 440 Hz and the patient is requested to adapt the amplitude of the played sound in such a way that the sound is just as loud as the dominant sound of his ringing in the ear. The patient is questioned whether the dominant sound of his ringing in the ear lies lower or higher than the frequency of the played sound. If the dominant sound of the ringing in the ear is higher, an upwardly directed arrow is then introduced into the column provided for step 1 in the table shown in FIG. 2. If the dominant sound of the ringing in the ear is lower, a downwardly directed arrow is then introduced into the table. The process is repeated with the next frequency of 550 Hz and also of the subsequent frequency of 700 Hz.

When the patient states that the dominant sound of the ringing in the ear is lower than a sound from the lower spectrum (440 Hz, 550 Hz, 700 Hz, . . . ), this means that when the arrow turns its direction (from upwards to downwards) the test is terminated for the lower spectrum.

For limiting the spectrum upwardly one starts with the highest frequency of 14,000 Hz. The sound is played to the patient and the patient is asked to match the amplitude of a sound in such a way that the volume of the external sound corresponds to the amplitude of the dominant sound of the ringing in the ear. The patient is asked whether the frequency of the dominant sound of the ringing in the ear is lower or higher than the frequency of the external sound. If the dominant sound of the ringing in the ear is higher an upwardly directed arrow is introduced into the column provided for step 1 of the table shown in FIG. 2. If the dominant sound of the ringing in the ear is lower a downwardly directed arrow is introduced into the table.

The process is, as previously described, repeated with the second highest frequency of 12,500 Hz and subsequently with the third highest frequency of 11,200 Hz.

When the patient determines that the dominant sound of the ringing in the ear is higher than the sound from the upper spectrum (14,000 Hz, 12,500 Hz, 11,200 Hz, . . . ), this means when the arrow direction is reversed (from downwards to upwards) then the test is terminated for the upper spectrum.

As an example for this measurement exemplary arrows for the frequency comparison are input into the table illustrated in FIG. 3.

As a result the step 1 provides two numbers the lower threshold and the upper threshold. In the example of FIG. 3, the lower threshold amounts to 880 Hz and the upper threshold 5,600 Hz, this means that the frequency precisely before the change of direction of the arrow is determined.

Once the frequency spectrum has been limited in step 1 a similarity measurement is carried out in step 2. In this step test sounds are played, wherein the patient should state the similarity with respect to the frequency of the dominant sound of his ringing in the ear. For this purpose an exemplary scale of 0 to 10 is made available −0 means no similarity in the sense that both sounds are completely different, and 10 means a large similarity in the sense that both sounds are identical exists. Before the similarity can be evaluated, the amplitude of the test sound is to be set to the same amplitude of the dominant sound of the ringing in the ear of the patient. The step size is either a whole sound (lower part of the frequency scale, approximately 12% relative difference) or half a sound (upper part of the frequency scale, approximately 6% relative difference).

Initially the method and its procedure shall be explained to the patient.

One subsequently starts with the lowest frequency within the limitation (880 Hz). The sound is played and the patient is asked to set the amplitude of the sound in such a way that the external sound is just as loud as the dominant sound of the ringing in the ear of the patient. The patient is asked how similar this external sound is to the dominant sound of his ringing in the ear. The patient is asked to state this with reference to the scale of 0 to 10, wherein 0 means no similarity and 10 means complete conformity. The evaluation of the patient is inserted into the corresponding box of the table with an “X”.

The previous process is repeated with a different sound within the limitation, e.g. 2,800 Hz. One carries on for so long up until similarity values are determined for all sounds and are introduced into the table.

If the patient is not sure with respect to a similarity value, the similarity measurement is then repeated for the corresponding sound and the old similarity value is possibly replaced by a new similarity value.

An example for such a similarity measurement is illustrated in FIG. 3, there exemplary similarity values are introduced with “X”. In this example test sounds were used within the limitation of 880 Hz to 5,600 Hz determined in step 1. All other sounds that are smaller than 880 Hz or larger than 5,600 Hz have been cancelled. For each test sound the similarity is stated with a “X”. In the present example the test sound of 3,140 Hz has the highest similarity value to the dominant sound of the ringing in the ear. The determination of this frequency is the result of the step 2.

In the following step 3 a pairwise sound comparison is carried out in this work step pairs of sound are played for the patient at a more strongly differentiated scale, wherein the patient shall decide which of the respective two sounds is more similar to the dominant sound of the ringing in the ear perceived by him with respect to the pitch. For this purpose, for example, eight sounds are selected that are grouped around the sound having the highest similarity value determined in the step 2 (in the exemplary table of FIG. 3 this is the sound of 3,140 Hz) and are then combined to pairs. The step size is either half a sound (lower part of the frequency scale, approximately 6% relative difference) or a quarter sound (upper part of the frequency scale, approximately 3% relative difference).

Initially the method and its procedure are explained to the patient.

In a first pairwise sound comparison eight sounds are pairwise compared to one another that are referred to in the first column of step 3 in the table of FIG. 3 with A1, B1, C1, D1, A2, B2, C2, D2 and which stem from the frequency range of 2,640 Hz to 3,950 Hz.

The first sound pair is formed from the first and the fifth sound, which are referred to in FIG. 3 with A1 respectively A2. The sound A1 (2,640 Hz) is played and the patient is requested to set the amplitude of this sound in such a way that the external sound is just as loud as the dominant sound of the ringing in the ear perceived by him. This sound is then named “sound 1”.

Furthermore, the sound A2 (3,320 Hz) is played for the patient and the patient is again requested to set the amplitude of this sound in such a way that the sound is just as loud as the dominant sound of the ringing in the ear. This sound is termed “sound 2”.

A change is made between the sounds wherein the sounds are respectively always termed “sound 1” respectively “sound 2”. The patient is asked which sound is more similar to the dominant sound of his ringing in the ear. The more similar sound is marked in the table with “+” and the less similar sound is marked with “−”.

The second pair is formed from the second and the sixth sound which are referred to in FIG. 3 with B1 respectively B2. The second sound pair is investigated in the same way as the first sound pair.

Subsequently one proceeds as described above with the third sound pair (C1 and C2) and the fourth sound pair (D1 and D2).

In the first pairwise sound comparison four sounds were determined that are marked with “+” in the first column of step 3 in FIG. 3.These sounds are used for the purpose of forming the sound pairs for the second pairwise sound comparison. All other sounds are cancelled out.

The first sound pair of the pairwise sound comparison is composed of the “winners” of A1/A2 and C1/C2 of the first pairwise sound comparison. In the exemplary table of FIG. 3 these are the sounds with 2,960 Hz and 3,320 Hz. The investigation of this sound pair takes place in the same way as described above.

The second sound pair of the second pairwise sound comparison is composed of the “winners” of B1/B2 and D1/D2 of the first pairwise sound comparison. In the exemplary table of FIG. 3 these are the sounds with 3,140 Hz and 3,500 Hz. The investigation of this sound pair now takes place in the same way as described above.

As a result of the second pairwise sound comparison, two sounds were marked with “+”. In the exemplary table of FIG. 3 these are the sounds with 3,320 Hz and 3,500 Hz. These two sounds form the pair for the third and the last pairwise sound comparison. The investigation of the sound pair is again carried out in the same way to the previously described comparison investigations.

The winner of the last comparison in the exemplary table illustrated in FIG. 3 is the sound with 3,320 Hz, and it is the result of step 3.

It is furthermore plausible to deviate from the method previously described in step 3. Rather than letting the patient decide between two sounds that are played, the sound pair, a similarity measurement for each of the two sounds can also be carried out in step 2. The sound having a respectively higher similarity value is then used for the purpose of forming the next pair and the sound having the respectively lowest value is cancelled for the next comparison stage.

In step 4 finally a fine adjustment of the external sound within narrowly set boundaries is carried out in order to achieve an as large as possible approximation of the dominant sound of the ringing in the ear. Within set boundaries in this respect means ±5%. Correspondingly for the present example a fine adjustment for the interval of 3,140 to 3,500 Hz which are the frequencies of the two adjacent sounds of step 3 is carried out. The step size within this interval is flexible (approximately 1% accuracy).

Initially the method and its procedure are explained to the patient.

Thereupon a sound is played with the frequency determined in step 3 (in the table of FIG. 3 this is 3,320 Hz) and the patient is requested to set the amplitude of this sound in such a way that it corresponds to the amplitude of the dominant sound of the ringing in the ear perceived by him.

Subsequently the patient can set the frequency of the sound within the set boundary, for example with the aid of a rotary button. The patient should terminate the fine adjustment as soon as the external sound is ideally set to the frequency of the dominant sound of the ringing in the ear. This frequency is inserted into the table at step 4 as a result of the method described herein. In the exemplary table illustrated in FIG. 3 the frequency determined in step 4 amounts to 3,290 Hz.

1-16. (canceled)

17. An apparatus for determining a frequency of at least one dominant tone of a tinnitus perceived by a patient, the apparatus comprising:
a sound generator configured to generate a plurality of tones of different frequencies;
an input unit configured to receive input evaluations from the patient relating to the generated plurality of tones; and
a control and analysis unit coupled to the sound generator and the input unit, with the control and analysis unit being configured to execute a similarity measurement and a pairwise tone comparison and to further combine results of the similarity measurement and the pairwise tone comparison to determine at least one tone having a frequency that is most similar to a frequency of at least one dominant tone of the tinnitus perceived by the patient,
wherein, for the similarity measurement, the control and analysis unit is configured to control the sound generator to it successively generate the plurality of tones,
after a respective one of the plurality of tones has been generated, receive a first input evaluation that is input by the patient into the input unit being indicative of a similarity of the generated respective one of the plurality of tones to the at least one dominant tone of the tinnitus, and
as a result of the similarity measurement, determine one reference tone based on the received first input evaluation, the frequency of which is most similar to the frequency of the at least one dominant tone of the tinnitus, and
wherein, for the pairwise tone comparison, the control and analysis unit is further configured to:
control the sound generator to successively generate pairs of tones that lie within a predefined frequency range around the frequency of the reference tone determined as a result of the similarity measurement, and
after a respective pair of tones has been generated, receive a second input evaluation that is input by the patient into the input unit being indicative about which one of the respective pair of tones is more similar to the at least one dominant tone of the tinnitus.

18. The apparatus in accordance with claim 17, wherein the control and analysis unit is configured to control the sound generator to generate the plurality of tones by selecting from a set of predefined tones, respectively, wherein a frequency spacing of adjacent predefined tones for the similarity measurement is larger than a frequency spacing of adjacent predefined tones for the pairwise tone comparison.

19. The apparatus in accordance with claim 17, wherein the control and analysis unit is further configured to determine, based on the pairwise tone comparison, at least one tone based on the second input evaluation that has a frequency that is most similar to the frequency of the at least one dominant tone of the tinnitus.

20. The apparatus in accordance with claim 17, wherein the control and analysis unit is configured to select, for the pairwise tone comparison, the respective tones of the generated pairs of tones that, based on the second evaluation input, are more similar to the at least one dominant tone of the tinnitus, wherein the control and analysis unit is further configured to control the sound generator to generate subsequent pairs of tones that are based on a combination of tones previously selected by the control and analysis unit.

21. The apparatus in accordance with claim 17, wherein the first input evaluation input by the patient indicates an evaluation by the patient of the similarity of the generated tones to the at least one dominant tone of the tinnitus based on a psychophysical similarity measure.

22. The apparatus in accordance with claim 17, wherein the first input evaluation input by the patient indicates an evaluation by the patient of the similarity of the generated tones to the at least one dominant tone of the tinnitus with respect to at least one of a pitch of the tone, a volume of the tone and a characteristics of the tone.

23. The apparatus in accordance with claim 17, wherein the second input evaluation input by the patient indicates an evaluation by the patient of the similarity of the generated tones to the at least one dominant tone of the tinnitus based on a psychophysical similarity measure.

24. The apparatus in accordance with claim 17, wherein the second input evaluation input by the patient indicates an evaluation by the patient of the similarity of the generated tones to the at least one dominant tone of the tinnitus with respect to at least one of a pitch of the tone, a volume of the tone and a characteristics of the tone.

25. The apparatus in accordance with claim 17, wherein the control and analysis unit is configured to control the sound generator such that a respective tone is generated for the similarity measurement that depends on the first input evaluation associated to a previously generated tone.

26. The apparatus in accordance with claim 17, wherein the control and analysis unit is configured to control the sound generator such that a respective pair of tones is generated for the pairwise comparison that depends on the second input evaluation associated to a previously generated pair of tones.

27. The apparatus in accordance with claim 17, wherein the control and analysis unit is further configured to:
receive a volume level to be set input by the patient into the input unit and that is indicative of a volume level of the at least one dominant tone of the tinnitus, and
control the sound generator based on the volume level to be set such that the sound generator generates at least one tone having a volume level that corresponds to the volume level of the at least one dominant tone of the tinnitus perceived by the patient, and
control the sound generator such that a volume level of the tones generated for at least one of the similarity measurement and the pairwise tone comparison is matched to the volume level of the at least one dominant tone.

28. The apparatus in accordance with claim 17,
wherein the control and analysis unit is further configured to perform a coarse estimate of the frequency of the at least one dominant tone of the tinnitus perceived by the patient, for which purpose the control and analysis unit controls the sound generator such that the sound generator generates the plurality of tones one after another, and
wherein the control and analysis unit receives the respective input evaluations input by the patient into the input unit after a respective tone has been generated and being indicative of whether the frequency of the respective tone is larger or smaller than the frequency of the at least one dominant tone of the tinnitus.

29. The apparatus in accordance with claim 28, wherein, based on the coarse estimate of the frequency of the at least one dominant tone of the tinnitus perceived by the patient, the control and analysis unit is further configured to determine the predefined frequency range and to control the sound generator for at least one of the similarity measurement and the pairwise tone comparison such that the sound generator generates the plurality of tones or pairs of tones from the predefined frequency range.

30. A method for determining a frequency of at least one dominant tone of a tinnitus perceived by a patient, the method comprising:
performing a similarity measurement that includes:
successively playing, by a sound generator, a plurality of tones of different frequencies to the patient,
after playing a respective one of the plurality of tones, receiving a first input evaluation of the patient that is indicative of a similarity of the played respective one of the plurality of tones to the at least one dominant tone of the tinnitus, and
determining, as a result of the similarity measurement, one reference tone based on the received first input evaluation, the frequency of which is most similar to the frequency of the at least one dominant tone of the tinnitus;
performing a pairwise tone comparison that includes:
successively playing pairs of tones that lie within a predefined frequency range around the frequency of the reference tone determined as a result of the similarity measurement, and
after playing a respective pair of tones, receiving a second input evaluation of the patient that is indicative about which one of the respective pair of tones is more similar to the at least one dominant tone of the tinnitus; and
combining results of the similarity measurement and the pairwise tone comparison to determine at least one tone having a frequency that is most similar to the frequency of the at least one dominant tone of the tinnitus.

31. The method in accordance with claim 30, further comprising generating, by the sound generator, the plurality of tones by selecting from a set of predefined tones, respectively, wherein a frequency spacing of adjacent predefined tones for the similarity measurement is larger than a frequency spacing of adjacent predefined tones for the pairwise tone comparison.

32. The method in accordance with claim 30, further comprising determining, based on the pairwise tone comparison, at least one tone based on the second input evaluation that has a frequency that is most similar to the frequency of the at least one dominant tone of the tinnitus.

33. The method in accordance with claim 30, further comprising:
receiving a volume level to be set input by the patient and that is indicative of a volume level of the at least one dominant tone of the tinnitus;
controlling the sound generator based on the volume level to be set such that the sound generator generates at least one tone having a volume level that corresponds to the volume level of the at least one dominant tone of the tinnitus perceived by the patient; and
controlling the sound generator such that a volume level of the tones generated for at least one of the similarity measurement and the pairwise tone comparison is matched to the volume level of the at least one dominant tone.

34. The method in accordance with claim 30, further comprising:
performing a coarse estimate of the frequency of the at least one dominant tone of the tinnitus perceived by the patient to control the sound generator to generate the plurality of tones one after another; and
receiving the respective input evaluations input by the patient after a respective tone has been generated and being indicative of whether the frequency of the respective tone is larger or smaller than the frequency of the at least one dominant tone of the tinnitus.

35. The method in accordance with claim 34, further comprising:
determining, based on the coarse estimate of the frequency of the at least one dominant tone of the tinnitus perceived by the patient, the predefined frequency range; and
controlling the sound generator for at least one of the similarity measurement and the pairwise tone comparison to generate the plurality of tones or pairs of tones from the predefined frequency range.

36. A computer program product for execution in a data processing system, the computer program product including instructions for performing a method for determining a frequency of at least one dominant tone of a tinnitus perceived by a patient, the method comprising:
performing a similarity measurement that includes:
successively playing a plurality of tones of different frequencies to the patient,
after playing a respective one of the plurality of tones, receiving a first input evaluation of the patient that is indicative of a similarity of the played respective one of the plurality of tones to the at least one dominant tone of the tinnitus, and
determining, as a result of the similarity measurement, one reference tone based on the received first input evaluation, the frequency of which is most similar to the frequency of the at least one dominant tone of the tinnitus;
performing a pairwise tone comparison that includes:
successively playing pairs of tones that lie within a predefined frequency range around the frequency of the reference tone determined as a result of the similarity measurement, and
after playing a respective pair of tones, receiving a second input evaluation of the patient that is indicative about which one of the respective pair of tones is more similar to the at least one dominant tone of the tinnitus; and
combining results of the similarity measurement and the pairwise tone comparison to determine at least one tone having a frequency that is most similar to the frequency of the at least one dominant tone of the tinnitus.
